(19) United States
(12) Patent Application Publication
(10) Pub. No.: US 2018/0140579 A1
(43) Pub. Date: May 24, 2018

(54) SACUBITRIL AND VALSARTAN FOR TREATING METABOLIC DISEASE

(71) Applicant: NOVARTIS AG, Basel (CH)

(72) Inventors: Diego ALBRECHT, Basel (CH); Thomas LANGENICKEL, Bottmingen (CH); Jens JORDAN, Hannover (DE); Stefan ENGELI, Hannover (DE)

(21) Appl. No.: 15/577,042

(22) PCT Filed: May 27, 2016
(86) PCT No.: PCT/IB2016/053128

§ 371 Date: Nov. 27, 2017

Related U.S. Application Data
(60) Provisional application No. 62/168,005, filed on May 29, 2015.

(51) Int. Cl. A61K 31/41; A61K 31/216; A61P 3/10; A61P 3/06

(52) U.S. Cl. CPC A61K 31/41; A61K 31/216; A61K 2300/00; A61P 3/06; A61P 3/10

(57) ABSTRACT

The present invention relates to methods and pharmaceutical compositions for prevention or treatment of a metabolic disease in a human patient in need thereof, comprising administration of a therapeutically effective amount, or a prophylactically effective amount, of a combination of sacubitril and valsartan in a 1:1 molar ratio to said patient.

The present invention relates to methods and pharmaceutical compositions for the prevention or treatment of a metabolic disease in a human patient in need thereof, comprising administration of a therapeutically effective amount, or a prophylactically effective amount, of a combination of sacubitril or a pharmaceutically acceptable salt thereof and valsartan or a pharmaceutically acceptable salt thereof in a 1:1 molar ratio to said patient.

### BACKGROUND OF THE INVENTION

### Metabolic Diseases—Obesity and Metabolic Syndrome

Overweight, adiposity and obesity are conditions defined as abnormal or excessive fat accumulation that may impair health. It results from imbalances in the body's regulation of energy intake, expenditure and storage.

Obesity is a public health issue in the United States with more than one third of the adult population being identified as obese. Obesity in children is also on the rise. Obesity is also associated with an increased risk of a variety of co-morbid conditions such as diabetes, atherosclerosis and hypertension. Obesity also is one of the leading risk factors for metabolic syndrome. Metabolic syndrome is a group of five risk factors that increase an individual's risk for heart disease and other health problems such as diabetes and stroke. The five conditions or risk factors are high blood pressure, low HDL cholesterol levels in blood, large waistline, high triglyceride levels in blood, and high fasting blood sugar. Individuals with three or more of these conditions are diagnosed with metabolic syndrome.

Metabolic syndrome (also called syndrome X) is becoming an increasingly common diagnosis as the obesity rates rise in the United States. People with the metabolic syndrome are also at increased risk for cardiovascular disease and for increased mortality from both cardiovascular disease and all causes. Accordingly, health professionals predict that, sometime in the near future, metabolic syndrome may overtake smoking as the leading risk factor for heart disease. Metabolic syndrome is a complex syndrome which can be associated with several of following criteria such as resistance to insulin-stimulated glucose uptake, glucose intolerance, hyperinsulinemia, increase LDL-cholesterol, increased VLDL triglycerides, decreased HDL cholesterol, hypertriglyceridemia, and others. It is known that one of the diseases that commonly develops in patients with metabolic syndrome is type II diabetes; in turn, the number one cause of death of patients with type II diabetes is atherosclerosis, a disease that causes plaques to build up in the arteries and eventually lead to heart attacks and stroke.

As a result, there is a great deal of interest in identifying new targets for development of therapies to prevent and treat obesity (adiposity), metabolic syndrome, and the conditions that can develop as a result, such as insulin resistance and cardiovascular diseases.

### Cardiovascular Diseases

Chronic heart failure (CHF) is a major public health problem characterized by significant mortality, frequent hospitalization, and poor quality of life, with an overall prevalence that is increasing throughout the world. In the United States (US) alone, approximately 5 million patients have heart failure (HF) and there are over half a million newly diagnosed cases annually. In Europe, the prevalence of HF is between 2 and 3%, and that in the elderly is estimated between 10 to 20%.

Heart failure is characterized by neurohormonal imbalance in favor of activation of the renin-angiotensin-aldosterone system (RAAS) which is contrasted by a relative deficiency of endogenous protective systems with opposing effects such as the natriuretic peptide (NP) system. This neurohormonal pattern is thought to contribute to changes in peripheral metabolic function including impaired oxidative capacity, which predisposes to insulin resistance through accumulation of toxic lipid intermediates and may further limit exercise capacity and contribute to poor clinical outcomes.

Medical therapies targeted at improving outcomes in HF with a low LVEF have been well studied over the past two decades, leading to an improvement in survival as well as a decrease in morbidity, mostly in the form of decrease in re-hospitalization for HF. These medical therapies include angiotensin converting enzyme (ACE) inhibitors, angiotensin receptor blockers (ARBs), β-blockers and mineralocorticoid antagonists.

However, despite advances in pharmacological (and device therapies), the outlook remains poor. Overall 50% of patients die within 4 years, and 40% of patients admitted to hospital with HF die or are readmitted within 1 year. Thus, HF still represents a major cause of cardiac mortality and morbidity with a clear need for better therapy.

Hypertension is a also major public health problem: Approximately 333 million adults in economically developed countries and about 65 million Americans (1 in 3 adults) had high blood pressure in 2000. Prolonged and uncontrolled hypertensive vascular disease ultimately leads to a variety of pathological changes in target organs, such as the heart and kidney. Sustained hypertension can lead as well to an increased occurrence of stroke. Hypertension includes and is not limited to mild, moderate and severe hypertension as defined in Journal of Hypertension 1999, 17:151-183, especially on page 162. Isolated systolic hypertension (ISH) is the most common form of hypertension in people over 50 years. It is defined as elevated systolic blood pressure (above 140 mm Hg) in conjunction with normal diastolic blood pressure (below 90 mm Hg). Elevated systolic blood pressure is an independent risk factor for cardiovascular diseases and may lead e.g. to myocardial hypertrophy and heart failure. ISH is furthermore characterized by an increased pulse pressure, defined as the difference between systolic and diastolic blood pressures. Elevated pulse pressure is being recognized as the type of hypertension the least likely to be well controlled.

### Sacubitril and Valsartan (LCZ696)

LCZ696 is a first-in-class angiotensin receptor neprilysin inhibitor (ARNI) being developed for the treatment of cardiovascular diseases such as hypertension and/or heart failure. Ingestion of LCZ696 results in systemic exposure to sacubitril (AHU377; (2R,4S)-5-biphenyl-4-yl-4-(3-carboxy-propionylamino)-2-methyl-pentanoic acid ethyl ester, also named N-(3-carboxy-1-oxopropyl)-(4S)-(p-phenylphenylmethyl)-4-amino-2R-methylbutanoic acid ethyl ester), a neprilysin (neutral endopeptidase 24.11, NEP) inhibitor (NEPi) prodrug which is converted to the active form LBQ657 (2R,4S)-5-biphenyl-4-yl-4-(3-carboxy-propionyl amino)-2-methyl-pentanoic acid), and valsartan providing inhibition of the angiotensin II type 1 (AT1) receptor, in a 1:1 molar ratio.

Neprilysin (NEP) inhibition will expose subjects to enhanced levels of the physiologically active natriuretic peptides (NPs), including atrial natriuretic peptide (ANP). NPs mediate their cardiovascular effects through activation of the natriuretic peptide receptor A (NPR-A) and their second messenger cyclic GMP (cGMP) resulting in potent vasodilation, natriuresis and diuresis, inhibition of the renin angiotensin aldosterone system (RAAS) by reducing renin and aldosterone release, reduced sympathetic drive, and anti-proliferative and anti-hypertrophic effects on vascular endothelium and smooth muscle cells. The angiotensin receptor blocker (ARB) blockade is specific and competitive at the angiotensin type 1 (AT1) receptor that mediates the deleterious effects of angiotensin II on the cardiovascular system. LCZ696 provides concomitant NEP inhibition and AT1 blockade, which are considered to act complementary. Thus, LCZ696 may deliver clinical benefits to patients with cardiovascular disease, including heart failure and hypertension, in which vasoconstriction, volume expansion, and target organ damage play a key role in pathophysiology.

The compounds and pharmaceutical compositions disclosed herein include combinations of sacubitril and valsartan which compounds or pharmaceutical compositions thereof have been previously disclosed in WO 2003/059345, WO 2007/056546, WO 2009/061713, and WO2014029848, which are herein incorporated by reference.

The effects of LCZ696 on insulin sensitivity are unknown. However, angiotensin II type 1 (AT1) receptor blockade (ARB) has been shown to ameliorate insulin sensitivity (Jing et al 2012). Among patients with impaired glucose tolerance and cardiovascular disease or risk factors, the use of valsartan for 5 years, along with lifestyle modification, led to a relative reduction of 14% in the incidence of diabetes (McMurray 2010). In contrast to angiotensin II, knowledge on the role of NPs in glucose metabolism is limited. A short term cross over study in healthy subjects indicated that intravenous BNP decreases circulating glucose concentrations after glucose load, without affecting insulin secretion and clearance (Heinisch 2011). In addition, obese individuals and individuals with metabolic syndrome have lower NP plasma levels which are inversely related to left ventricular hypertrophy and metabolic risk factors (Wang 2007, Beleigoli 2009), suggesting that lower NP levels may contribute to the incidence of adverse cardiovascular events in these individuals (Rubattu 2008). Also, individuals with reduced circulating NP levels are prone to progress to type 2 diabetes mellitus (Magnusson et al 2012).

Furthermore, ANP potently promotes lipid mobilization from adipose tissue (Sengenes et al 2000, Birkenfeld et al 2005, Souza et al 2011), increases postprandial lipid oxidation (Birkenfeld et al 2008), elicits adiponectin release (Birkenfeld et al. 2012), and enhances oxidative capacity of human skeletal muscle cells (Engeli et al. 2012). Indeed, mice chronically overexpressing components of the natriuretic peptide system are protected from diet-induced obesity and insulin resistance (Miyashita et al 2009).

However, the net effect of ANP on lipolysis and oxidative metabolism of fatty acids and its clinical implications is unknown. Although angiotensin II and ANP have been implicated in the regulation of glucose and free fatty acid metabolism and may suggest overall favorable effects of the combination of an angiotensin receptor blocker and a neprilysin inhibitor (i.e. an angiotensin receptor neprilysin inhibitor (ARNI)) at rest and during exercise, the net metabolic effects of long-term treatment with such an ARNI in a population at metabolic risk are unknown.

Overall there remains a great need in identifying new targets for development of therapies to treat obesity (adiposity), metabolic syndrome, and the conditions that can develop as a result, such as insulin resistance and cardiovascular diseases. In particular there is a need for the development of new therapies for the treatment of metabolic syndrome, like insulin resistance in patients suffering from obesity (adiposity) and cardiovascular disorders such as hypertension.

### SUMMARY OF THE INVENTION

Surprisingly, the administration of a pharmaceutical composition comprising a therapeutically effective amount, or a prophylactically effective amount, of an Angiotensin Receptor Neprilysin inhibitor (ARNi) as defined herein or of a therapeutically effective amount, or a prophylactically effective amount, of a combination of the Angiotensin Receptor Blocker (ARB) valsartan with the Neutral Endopeptidase inhibitor (NEPi) sacubitril in a 1:1 molar ratio, as defined herein, to patients in need thereof has been shown to favorably impact glucose metabolism in obese hypertensive subjects. Accordingly, the therapeutic approach resulting in simultaneous ARB and enhancement of the NP system has been shown to exert beneficial metabolic effects in patients with cardiovascular diseases, including HF. In particular, it has been shown that LCZ696 improves insulin sensitivity and lipid mobilization from subcutaneous abdominal adipose tissue in comparison to the metabolically neutral comparator amlodipine.

Accordingly, in a first aspect the present invention relates to a method for the prevention or treatment of a metabolic disease in a human patient in need of such prevention or treatment comprising administering to said patient a pharmaceutical composition comprising a therapeutically effective amount or a prophylactically effective amount of a combination of sacubitril and valsartan in a 1:1 molar ratio.

In one embodiment thereof, the patient also suffers from a cardiovascular disease.

Said pharmaceutical composition comprises a combination of a 1:1 molar ratio of
(i) valsartan or a pharmaceutically acceptable salt thereof; and
(ii) sacubitril or a pharmaceutically acceptable salt thereof.

In one embodiment, said combination is provided in the form of the compound of the formula (I)

[(A₁)(A₂)](Na⁺)_{y}.xH₂O (I)

wherein
A₁ is valsartan in the anionic form;
A₂ is sacubitril in the anionic form;
Na⁺ is a sodium ion;
y is 1 to 3; and
x is 0 to 3.

In one embodiment thereof, the compound of formula (I) is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

In one embodiment, the pharmaceutical composition comprises in addition one or more pharmaceutically acceptable carriers.

In a second aspect, the present invention is directed to the use of the pharmaceutical composition as defined above for the manufacture of a medicament for the prevention or treatment of a metabolic disease in a human patient, preferably a patient also suffering from a cardiovascular disease.

In a third aspect, the present invention is directed to a pharmaceutical composition as defined above for use in the prevention or treatment of a metabolic disease in a human patient, preferably a patient also suffering from a cardiovascular disease.

In a fourth aspect, the present invention is directed to the use of a pharmaceutical composition as defined above in the prevention or treatment of a metabolic disease in a human patient, preferably a patient also suffering from a cardiovascular disease.

### Definitions

Throughout this specification and in the claims that follow, the following terms are defined with the following meanings, unless explicitly stated otherwise.

The term “prevention” refers to prophylactic administration to a healthy subject to prevent the development of the conditions mentioned herein. Moreover, the term “prevention” means prophylactic administration to patients being in a pre-stage of the conditions to be treated.

The term “treatment” is understood the management and care of a patient for the purpose of combating the disease, condition or disorder.

The term “therapeutically effective amount” refers to an amount of a drug or a therapeutic agent that will elicit the desired biological and/or medical response of a tissue, system or an animal (including man) that is being sought by a researcher or clinician.

The terms “patient” include, but are not limited to, humans, dogs, cats, horses, pigs, cows, monkeys, rabbits and mice. The preferred patients are humans.

The terms “administration of” and or “administering a” compound should be understood to mean providing a compound of the invention or a pharmaceutically acceptable salt or ester thereof, or a pro-drug thereof to a subject in need of treatment. The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well-known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “prophylactically effective amount” as used herein means the amount of the active compounds in the composition that will elicit the biological or medical response in a tissue, system, subject, or human that is being sought by the researcher, veterinarian, medical doctor or other clinician, to prevent the onset of a disease characterized and/or manifested by atrial enlargement and/or remodeling.

As used herein, the term “about” refers to +/−20%, +/−10%, or +/−5% of a value.

The term “pharmaceutically acceptable”, as used herein, refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

In the context of the present invention, the term “sacubitril and valsartan in a 1:1 molar ratio” refers to an Angiotensin Receptor Neprilysin inhibitor (ARNi) which is
a) trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696), or
b) a combination comprising a therapeutically effective amount of a 1:1 molar ratio of
   (i) valsartan or a pharmaceutically acceptable salt thereof; and
   (ii) sacubitril or a pharmaceutically acceptable salt thereof.

The term “metabolic disease,” and terms similarly used herein, includes but is not limited to adiposity (obesity), insulin resistance, impaired glucose metabolism, impaired glucose tolerance (IGT), hyperglycemia, conditions of impaired fasting plasma glucose, metabolic syndrome, diabetes mellitus type II; hyperinsulinemia, hyperlipidemia, dyslipidemia, and hypertriglyceridemia.

“Obesity” or “Adiposity”, are used interchangeably in the context of the current invention. In terms of the human subject, they can be defined as an adult with a Body Mass Index (BMI) of 30 or greater (Centers for Disease Control and Prevention).

“Insulin resistance” is defined as a state in which a normal amount of insulin produces a subnormal biologic response.

“Glucose intolerance” or “Impaired Glucose Tolerance” (IGT) is a pre-diabetic state of dysglycemia that is associated with increased risk of cardiovascular pathology. The pre-diabetic condition prevents a subject from moving glucose into cells efficiently and utilizing it as an efficient fuel source, leading to elevated glucose levels in blood and some degree of insulin resistance. Glucose intolerance is characterized by a pathological state in which the fasting plasma glucose level is less than 140 mg per deciliter and the 30-, 60-, or 90-minute plasma glucose concentration following a glucose tolerance test exceeds 200 mg per deciliter.

“Hyperglycemia” is defined as an excess of sugar (glucose) in the blood.

“Hyperinsulinemia” is defined as a higher-than-normal level of insulin in the blood.

Hyperinsulinemia is caused by overproduction of insulin by the body and related to insulin resistance.

“Metabolic syndrome” can be defined as a cluster of at least three of the following signs: abdominal fat—in most men, a 40-inch waist or greater; high blood glucose—at least 110 milligrams per deciliter (mg/dl) after fasting; high triglycerides—at least 150 mg/dL in the bloodstream; low HDL—less than 40 mg/dl; and, blood pressure of 130/85 mmHg or higher.

Very low density lipoprotein (VLDL) are large lipoproteins rich in triglycerides which circulate through the blood giving up their triglycerides to fat and muscle tissue until the VLDL remnants are modified and converted into LDL.

High density lipoprotein (HDL) are lipoproteins that transport cholesterol in the blood; composed of a high proportion of protein and relatively little cholesterol; high levels are thought to be associated with decreased risk of coronary heart disease and atherosclerosis.

“Diabetes mellitus Type 2 (T2DM)” is a condition characterized by excess blood glucose concentration in spite of the availability of insulin.

“Dyslipidemia” is a disorder of lipoprotein metabolism, including lipoprotein overproduction or deficiency. Dyslipidemias may be manifested by elevation of the total cholesterol, low-density lipoprotein (LDL) cholesterol or triglyceride concentrations, or a decrease in high-density lipoprotein (HDL) cholesterol concentration in the blood.

“Hypertriglyceridemia” is defined by elevated triglyceride concentration in the blood.

“Hyperlipidemia” is characterized by the presence of excess lipids in the blood.

“Central obesity” or “Central adiposity” is characterized by the deposition of obesity around the trunk sparing the limbs. Central obesity is determined by measuring the waist-to-hip ratio. In one definition, central obesity is defined as a waist-to-hip ratio of >0.90 in men or >0.85 in women and/or body mass index (BMI) >30 kg/m2.

“Abdominal obesity” or “abdominal adiposity” is identified by measuring the waist circumference. In one embodiment, “Abdominal obesity” or “abdominal adiposity” are defined as a waist circumference >102 cm in men and >88 cm in women.

Among various known definitions of metabolic disease three of them are of particular relevance:

Metabolic syndrome is characterized by three or more of the following criteria:

1. Abdominal obesity: waist circumference >102 cm in men and >88 cm in women
2. Hypertriglyceridemia: ≥150 mg/dl (1.695 mmol/l)
3. Low HDL cholesterol: <40 mg/dl (1.036 mmol/l) in men and <50 mg/dl (1.295 mmol/l) in women
4. High blood pressure: ≥130/85 mmHg
5. High fasting glucose: ≥110 mg/dl (≥6.1 mmol/l).

Metabolic syndrome can also be characterized by three or more of the following criteria: triglycerides >150 mg/dl, systolic blood pressure (BP) ≥130 mm Hg or diastolic BP ≥85 mm Hg or on antihypertensive treatment, high-density lipoprotein cholesterol <40 mg/dl, fasting blood sugar (FBS) >110 mg/dl, and a BMI >28.8 k/m2.

Metabolic syndrome can also be characterized by diabetes, impaired glucose tolerance, impaired fasting glucose, or insulin resistance plus two or more of the following abnormalities:

1. High blood pressure: ≥160/90 mmHg
2. Hyperlipidemia: triglyceride concentration ≥150 mg/dl (1.695 mmol/l) and/or HDL cholesterol <35 mg/dl (0.9 mmol/l) in men and <39 mg/dl (1.0 mmol/l) in women
3. Central obesity: waist-to-hip ratio of >0.90 in men or >0.85 in women and/or body mass index (BMI) >30 kg/m2
4. Microalbuminuria: urinary albumin excretion rate ≥20 μg/min or an albumin-to-creatinine ratio ≥20 mg/g.

The New York Heart Association (NYHA) classification grades the severity of heart failure symptoms as one of four functional classes. The NYHA classification is widely used in clinical practice and in research because it provides a standard description of severity that can be used to assess response to treatment and to guide management. The New York Heart Association functional classification based on severity of symptoms and physical activity:
Class I: No limitation of physical activity. Ordinary physical activity does not cause undue breathlessness, fatigue, or palpitations.
Class II: Slight limitation of physical activity. Comfortable at rest, but ordinary physical activity results in undue breathlessness, fatigue, or palpitations.
Class III: Marked limitation of physical activity. Comfortable at rest, but less than ordinary physical activity results in undue breathlessness, fatigue, or palpitations.
Class IV: Unable to carry on any physical activity without discomfort. Symptoms at rest can be present. If any physical activity is undertaken, discomfort is increased.

### DETAILED DESCRIPTION OF THE INVENTION

This invention has shown based on the clinical trial described in more detail in the Example section that 8 weeks of treatment with LCZ696 improved insulin sensitivity and led to lipid mobilization from subcutaneous abdominal adipose tissue with unchanged whole body lipolysis and lipid oxidation at rest and during physical exercise in patients with hypertension and abdominal adiposity, thereby supporting for the first time the relevance of sustained elevation of neprilysin substrates such as natriuretic peptides in the context of AT1 receptor blockade to human glucose and lipid metabolism.

These findings imply that LCZ696 may improve cardiovascular and metabolic health in patients with cardiovascular disease. An inverse relationship between plasma natriuretic peptides and plasma glucose and insulin concentrations has been described in humans, and obese individuals; individuals with metabolic syndrome have lower natriuretic peptide plasma levels. LCZ696 is proposed to improve this relative deficiency of natriuretic peptides, thereby facilitating improvement of glucose and lipid metabolism.

The treated obese hypertensive patients can be seen as human model for impaired insulin sensitivity. Given the close association between adiposity and risk for type 2 diabetes, and the epidemic increase in the incidence of type 2 diabetes in recent years, cardiovascular medications with added benefits on insulin sensitivity are highly desirable.

In addition, it can be concluded that metabolic improvements with LCZ696 may also be beneficial in patients with heart failure, thereby differentiating LCZ696 from currently available cardiovascular drugs.

Accordingly, the present invention relates to the following:

### Methods of Treatment

The present invention is based upon the surprising and unexpected finding that certain drugs (i.e. LCZ696) effective for the treatment of cardiovascular disease or conditions, such as heart failure or hypertension, in human subjects in addition are able to provide beneficial effects on metabolic disease syndromes such as obesity and insulin resistance thereby enhancing the treatment potential for cardiovascular diseases.

Beneficial effects on metabolic disease syndromes was shown by improving insulin sensitivity and abdominal lipid mobilization inpatients with hypertension and abdominal adiposity, thereby supporting for the first time the relevance of sustained elevation of NPs to human glucose and lipid metabolism.

Thus, the invention encompasses a method for the prevention or treatment of a metabolic disease in a human patient in need of such prevention or treatment comprising administering to said patient a pharmaceutical composition comprising a therapeutically effective amount or a prophylactically effective amount of a combination of sacubitril and valsartan in a 1:1 molar ratio.

In one embodiment thereof, the metabolic disease is manifested by at least one of the following criteria selected from obesity, insulin resistance, impaired glucose metabolism, impaired glucose tolerance (IGT), conditions of impaired fasting plasma glucose, metabolic syndrome, diabetes mellitus type II; hyperglycemia, hyperinsulinaemia, hyperlipidaemia, abdominal adiposity, dyslipidemia, and hypertriglyceridemia.

In a preferred embodiment, the metabolic disease is manifested by at least one of the following criteria selected from insulin resistance, metabolic syndrome, and obesity/adiposity, in particular abdominal adiposity.

In another embodiment, sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

The present invention provides that the pharmaceutical composition comprising a therapeutically effective amount of sacubitril and valsartan in a 1:1 molar ratio is effective to provide prevention or treatment of a metabolic disease in a patient. The present invention also provides that the pharmaceutical composition is effective to induce at least one physiological effect in the mammal including improved insulin sensitivity and increased lipid mobilization from subcutaneous abdominal adipose tissue, in particular with unchanged whole body lipolysis.

The present invention also provides that the pharmaceutical composition better provides prevention or treatment of a metabolic disease than the corresponding amount of an calcium channel blocker, such as amlodipine, alone.

In one embodiment, the pharmaceutical composition better provides for the prevention, delay of progression or treatment of metabolic syndrome, wherein the metabolic syndrome is characterized by three or more of the following criteria:
1. Abdominal obesity: waist circumference >102 cm in men and >88 cm in women
2. Hypertriglyceridemia: ≥150 mg/dl (1.695 mmol/l)
3. Low HDL cholesterol: <40 mg/dl (1.036 mmol/l) in men and <50 mg/dl (1.295 mmol/l) in women
4. High blood pressure: ≥130/85 mmHg
5. High fasting glucose: ≥110 mg/dl (≥6.1 mmol/l)

In an another embodiment t the pharmaceutical composition better provides for the prevention, delay of progression or treatment of metabolic syndrome wherein the metabolic syndrome is characterized by diabetes, impaired glucose tolerance, impaired fasting glucose, or insulin resistance plus two or more of the following abnormalities:
1. High blood pressure: ≥160/90 mmHg
2. Hyperlipidemia: triglyceride concentration ≥150 mg/dl (1.695 mmol/l) and/or HDL cholesterol <35 mg/dl (0.9 mmol/l) in men and <39 mg/dl (1.0 mmol/l) in women
3. Central obesity: waist-to-hip ratio of >0.90 in men or >0.85 in women and/or BMI >30 kg/m2
4. Microalbuminuria: urinary albumin excretion rate ≥20 μg/min or an albumin-to-creatinine ratio ≥20 mg/g.

In an another embodiment the pharmaceutical composition better provides for the prevention, delay of progression or treatment of metabolic syndrome wherein the metabolic syndrome is characterized by three or more of the following: triglycerides >150 mg/dl, systolic blood pressure (BP) ≥130 mm Hg or diastolic BP ≥85 mm Hg or on antihypertensive treatment, high-density lipoprotein cholesterol <40 mg/dl, fasting blood sugar (FBS) >110 mg/dl, and a BMI >28.8 k/m2.

In one embodiment, the human patient also suffers from a cardiovascular disease.

In an embodiment thereof, the cardiovascular disease is selected from hypertension, chronic heart failure, acute heart failure, left ventricular dysfunction, endothelial dysfunction, diastolic dysfunction, ischemic cardiomyopathy, hypertrophic cardiomyopathy, diabetic cardiac myopathy, cardiac dysrhythmias, supraventricular and ventricular arrhythmias, atrial fibrillation, cardiac fibrosis, mitral stenosis and regurgitation, atrial flutter, detrimental vascular remodeling, heart target organ damage, plaque stabilization, myocardial infarction and its sequelae, atherosclerosis, angina pectoris, thrombosis, vascular aneurysm, vascular stenosis and infarction, vascular dementia, secondary aldosteronism, primary and secondary pulmonary hypertension, pulmonary congestion, pulmonary edema, right ventricular hypertrophy, and peripheral vascular disease.

In a further embodiment thereof, the patient also suffers from hypertension or heart failure.

In particular, the heart failure is congestive heart failure, left heart failure, right heart failure, chronic heart failure, advanced heart failure, acute heart failure, acute decompensated heart failure, heart failure with reduced ejection fraction, or heart failure with preserved ejection fraction.

In another embodiment thereof, the patient also suffers from heart failure, in particular chronic systolic heart failure with reduced ejection fraction (HF-rEF) or heart failure with preserved ejection fraction (HF-pEF).

In another embodiment of the foregoing, the patient suffering from chronic systolic heart failure, in particular the patient with chronic systolic heart failure with reduced ejection fraction, has at least one of the following characteristics:
i) heart failure of NYHA class II, III or IV,
ii) an elevated plasma BNP or NT-proBNP level, preferably a plasma BNP ≥100 pg/mL (or NT-proBNP ≥400 pg/mL), more preferably a plasma BNP ≥150 pg/mL or NT-proBNP ≥600 pg/mL, and
iii) a reduced left ventricular ejection fraction (LVEF) of ≤40%, preferably ≤35%.

In addition, the patient might be characterized by one or more of the following:
iv) prior hospitalization for heart failure within the last 12 months,
v) a stable ACE inhibitor or ARB at dose ≥enalapril 10 mg daily+beta-blocker (unless contraindicated or intolerant)+aldosterone antagonist (as indicated),
vi) systolic blood pressure ≥95 mm Hg,
vii) eGFR ≥30 ml/min/1.73 m² and
viii) serum K≤5.4 mEq/L.

In one embodiment, the patient also suffers from chronic heart failure classified as NYHA class II, III or IV and has systolic dysfunction. In particular, the patient has a reduced left ventricular ejection fraction (LVEF) of ≤40%, more particular ≤35%.

In another embodiment the patient has heart failure classified as NYHA class II. In a further embodiment, the patient has heart failure classified as NYHA class II with systolic dysfunction and has a reduced left ventricular ejection fraction (LVEF) of ≤40%, in particular ≤35%.

In another embodiment, the patient also suffers from hypertension, in particular mild to moderate essential hypertension.

In one embodiment, the present invention provides that the pharmaceutical composition comprising a therapeutically effective amount of sacubitril and valsartan in a 1:1 molar ratio is effective to induce at least one physiological effect selected from improving insulin sensitivity and increasing abdominal lipid mobilization in a patient suffering from a metabolic and a cardiovascular disease, in particular suffering from obesity (adiposity) and hypertension and/or heart failure.

In one embodiment, the present invention provides that the pharmaceutical composition comprising a therapeutically effective amount of sacubitril and valsartan in a 1:1 molar ratio is effective to ameliorate glucose metabolism in a patient suffering from a metabolic and a cardiovascular disease. In one embodiment thereof, the patient suffers from hypertension and abdominal adiposity. In another embodiment thereof, the patient suffers from heart failure and abdominal adiposity.

In another embodiment, the present invention provides that the pharmaceutical composition comprising a therapeutically effective amount of sacubitril and valsartan in a 1:1 molar ratio is effective to ameliorate glucose metabolism and to increase abdominal lipid mobilization in a patient suffering from a cardiovascular disease and adiposity, in particular in a patient suffering from hypertension and abdominal adiposity.

In one aspect of the present invention which applies to all of the aforementioned treatment options, the pharmaceutical composition is administered to deliver a daily overall dose of the combination of sacubitril and valsartan in a 1:1 molar ratio from about 50 mg to about 400 mg. In particular pharmaceutical composition is administered to deliver the combination of sacubitril and valsartan in a 1:1 molar ratio twice daily with a dose of 50 mg, 100 mg, or 200 mg.

In one embodiment thereof,
a) the 50 mg dose of sacubitril and valsartan in a 1:1 molar ratio corresponds to 24 mg sacubitril and 26 mg valsartan,
b) the 100 mg dose of sacubitril and valsartan in a 1:1 molar ratio corresponds to 49 mg sacubitril and 51 mg valsartan, and
c) the 200 mg dose of sacubitril and valsartan in a 1:1 molar ratio corresponds to 97 mg sacubitril and 103 mg valsartan.

In a particular embodiment of the pharmaceutical composition, the combination of sacubitril and valsartan in a 1:1 molar ratio is delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696), wherein
a) the 50 mg dose of sacubitril and valsartan in a 1:1 molar ratio corresponds to around 56.6 mg LCZ696,
b) the 100 mg dose of sacubitril and valsartan in a 1:1 molar ratio corresponds to around 113.1 mg LCZ696, and
c) the 200 mg dose of sacubitril and valsartan in a 1:1 molar ratio corresponds to around 226.2 mg LCZ696.

All the aforementioned embodiments for the methods of protection and treatment according to the present invention are equally applicable to
the use of the pharmaceutical compositions comprising a 1:1 molar ratio of sacubitril and valsartan as defined herein for the manufacture of a medicament for use according to the present invention,
the use of the pharmaceutical compositions comprising a 1:1 molar ratio of sacubitril and valsartan as defined herein according to the present invention,
the pharmaceutical compositions comprising a 1:1 molar ratio of sacubitril and valsartan as defined herein for use according to the present invention.

In particular, all the aforementioned embodiments for the methods of protection and treatment according to the present invention are equally applicable to the pharmaceutical compositions for the use in the prevention or treatment of a metabolic disease in a human patient according to the present invention, to the use of the pharmaceutical compositions for the prevention or treatment of a metabolic disease in a human patient according to the present invention and to the use of the pharmaceutical compositions for the manufacture of a medicament for the prevention or treatment of a metabolic disease in a human patient according to the present invention.

Some of these aspects are further described in more detail below, but this description should not be construed as limiting.

### Compounds and Compositions for Use According to the Invention

In the context of the present invention, the term “sacubitril and valsartan in a 1:1 molar ratio” refers to a combination comprising a therapeutically effective amount of a 1:1 molar ratio of
(i) valsartan or a pharmaceutically acceptable salt thereof; and
(ii) sacubitril or a pharmaceutically acceptable salt thereof.

Sacubitril is the INN for N-(3-carboxy-1-oxopropyl)-(4S)-(p-phenylphenylmethyl)-4-amino-2R-methylbutanoic acid ethyl ester. This is a prodrug for (2R,4S)-5-biphenyl-4-yl-4-(3-carboxy-propionyl amino)-2-methyl-pentanoic acid.

Valsartan is S—N-valeryl-N-{[2′-(1H-tetrazole-5-yl)-biphenyl-4-yl]-methyl}-valine.

In one embodiment thereof, the combination comprises a 1:1 molar ratio
(i) of valsartan; and
(ii) of N-(3-carboxy-1-oxopropyl)-(4S)-(p-phenylphenylmethyl)-4-amino-2R-methylbutanoic acid ethyl ester or a pharmaceutically acceptable salt thereof, such as sodium or calcium salt.

In another embodiment thereof, said combination is provided in the form of the compound of the formula (I)

[(A₁)(A₂)](Na⁺)_{y}.xH₂O (I)

wherein
A₁ is valsartan in the anionic form;
A₂ is sacubitril in the anionic form;
Na⁺ is a sodium ion;
y is 1 to 3, preferably 1, 2, or 3; and
x is 0 to 3, preferably 0, 0.5, 1, 1.5, 2, 2.5, or 3.

In one embodiment, y is 3 and x is 2.5.

In particular, the compound is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

In a preferred embodiment, the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate is present in crystalline form.

In a preferred embodiment, the invention encompasses a pharmaceutical composition for use comprising a therapeutically effective amount of trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (Compound LCZ696). Such compounds and pharmaceutical compositions have been previously disclosed in WO2007/056546 and WO 2009/061713, whose preparative teachings are incorporated herein by reference.

In a further embodiment of the invention, the pharmaceutical compositions for use according to the present invention comprise trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate] hemipentahydrate (LCZ696) and deliver upon administration the NEP inhibitor pro-drug and the angiotensin receptor blocker together to the patient.

In one embodiment of the invention for all of its uses, the pharmaceutical composition comprises the NEP inhibitor pro-drug sacubitril, namely N-(3-carboxy-1-oxopropyl)-(4S)-p-phenylphenylmethyl)-4-amino-(2R)-methylbutanoic acid ethyl ester or the NEP inhibitor N-(3-carboxy-1-oxopropyl)-(4S)-p-phenylphenylmethyl)-4-amino-(2R)-methylbutanoic acid, or pharmaceutically acceptable salts thereof, and the Angiotensin Receptor Blocker valsartan or a pharmaceutically acceptable salt thereof. Such combinations are for example disclosed within international patent application WO 2003/059345, which is herewith incorporated by reference.

In one embodiment, the pharmaceutical composition comprises the NEP inhibitor pro-drug sacubitril, namely N-(3-carboxy-1-oxopropyl)-(4S)-p-phenylphenylmethyl)-4-amino-(2R)-methylbutanoic acid ethyl ester or the NEP inhibitor N-(3-carboxy-1-oxopropyl)-(4S)-p-phenylphenylmethyl)-4-amino-(2R)-methylbutanoic acid, or pharmaceutically acceptable salts thereof, and the Angiotensin Receptor Blocker valsartan or a pharmaceutically acceptable salt thereof, in a 1:1 molar ratio.

(i) Valsartan or (S)—N-valeryl-N-{[2′-(1H-tetrazole-5-yl)-biphenyl-4-yl]-methyl}-valine) or a pharmaceutically acceptable salt thereof that can be purchased from commercial sources or can be prepared according to known methods, such as described in U.S. Pat. No. 5,399,578 and EP 0443983, whose preparative teachings are incorporated by reference herein. Valsartan may be used in certain embodiments of the invention in its free acid form, as well as in any suitable salt form. Depending upon the circumstance, esters or other derivatives of the carboxylic grouping may be employed as well as salts and derivatives of the tetrazole grouping.
(ii) sacubitril, namely N-(3-carboxy-1-oxopropyl)-(4S)-(p-phenylphenylmethyl)-4-amino-2R-methylbutanoic acid ethyl ester or (2R,4S)-5-biphenyl-4-yl-4(3-carboxy-propionyl amino)-2-methyl-pentanoic acid can be prepared by known methods such as described in U.S. Pat. No. 5,217,996 which is herein incorporated by reference.

The corresponding active ingredient or a pharmaceutically acceptable salt thereof may also be used in the form of a hydrate or include other solvents used for crystallization.

Preferably, the compounds sacubitril or a salt thereof, valsartan or a salt thereof, or LCZ696 are substantially pure or in a substantially pure form. As used herein, “substantially pure” refers to at least about 90% purity, more preferably at least about 95% and most preferably at least about 98% purity.

Also preferred is that these compounds are solid or a solid form or solid state. The solid, solid form or solid state can be crystalline, partially crystalline, amorphous or poly-amorphous, preferably in the crystalline form.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application.

The pharmaceutical preparations of the invention contain, for example, from about 0.1% to about 100%, e. g. 80% or 90%, or from about 1% to about 60%, of the active ingredient. The term “about” or “approximately”, as used herein in each instance, shall have the meaning of within 10%, more preferably within 5%, of a given value or range.

Pharmaceutical preparations according to the invention for enteral or parenteral administration are, e.g., those in unit dose forms, such as sugar-coated tablets, tablets, capsules, bars, sachets, granules, syrups, aqueous or oily suspensions or suppositories and furthermore ampoules. These are prepared in a manner known per se, e. g. by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

Tablets may be formed from the active compound with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tabletting the mixture by known methods. Similarly, capsules, for example hard or soft gelatin capsules, containing the active compound with or without added excipients, may be prepared by known methods. The contents of the capsule may be formulated using known methods so as to give sustained release of the active compound.

Other dosage forms for oral administration include, for example, aqueous suspensions containing the active compound in an aqueous medium in the presence of a non-toxic suspending agent such as sodium carboxymethylcellulose, and oily suspensions containing the active compounds in a suitable vegetable oil, for example arachis oil.

The active compound may be formulated into granules with or without additional excipients. The granules may be ingested directly by the patient or they may be added to a suitable liquid carrier (e.g. water) before ingestion. The granules may contain disintegrants, e.g. an effervescent pair formed from an acid and a carbonate or bicarbonate salt to facilitate dispersion in the liquid medium.

The dosage of the active ingredient of the composition will, of course, vary with the nature of the severity of the condition to be treated and with the particular compound in the composition and its route of administration. It will also vary according to the age, weight and response of the individual patient.

In the embodiments where the pharmaceutical composition comprises trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate] hemipentahydrate (LCZ696) in the pharmaceutical compositions for use in the context of the present invention, the unit dose of the therapeutic agents sacubitril and valsartan together will be in the range from about 1 to about 1000 mg, such as 40 mg to 400 mg (e.g., 50 mg, 100 mg, 200 mg, 400 mg) per day. Alternatively lower doses may be given, for example doses of 0.5 to 100 mg; 0.5 to 50 mg; or 0.5 to 20 mg per day. As explanatory note, a unit dose of 100 mg LCZ696 delivering 100 mg of the two agents sacubitril and valsartan corresponds to 113.1 mg of trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate]hemipentahydrate. Correspondingly, a unit dose of 50 mg requires 56.6 mg, a unit dose of 200 mg requires 226.2 mg, and a unit dose of 400 mg requires 452.4 mg of trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate]hemipentahydrate, respectively.

Dosages of the sum of the individual compounds sacubitril and valsartan or their respective salts in the combination of the pharmaceutical composition will be in the range from about 1 to about 1000 mg, such as 40 mg to 400 mg and include but are not limited to 5 mg, 20 mg, 25 mg, 40 mg, 50 mg, 80 mg, 100 mg, 200 mg, 400 mg, 800 mg and 1000 mg. Such dosages for the individual compounds sacubitril and valsartan can be considered therapeutically effective amounts or dosage strengths. Ratios for the amount of each compound in the pharmaceutical composition are preferably in the about 1:1 molar ratio to achieve an optimal renal protection while still providing cardiovascular benefits. Ratios for the amount of each compound in the pharmaceutical composition are preferably in the about 1:1 molar ratio to achieve an therapeutic effect for the metabolic disorders, while still providing cardiovascular benefits. In preferred embodiments, the dosages of the individual compounds sacubitril and valsartan correspond to the same molecular amounts as in a pharmaceutical composition comprising a 50 mg, 100 mg, 200 mg or 400 mg dose of LCZ696. E.g. a 200 mg dose of LCZ696 corresponds approximately to 103 mg valsartan and 97 mg of sacubitril.

Pharmaceutical compositions as used in the current invention can be administered any number of times per day, i.e. once a day (q.d.), twice (b.i.d.), three times, four time, etc. in an immediate release formation or less frequently as an extended or sustained release formation. Preferably the pharmaceutical composition is administered twice daily (b.i.d.). Corresponding doses may be taken, for example, in the morning, at mid-day or in the evening.

### Pharmaceutical Composition for Use

In a separate aspect, the present invention is directed to a pharmaceutical composition comprising a therapeutically effective amount or a prophylactically effective amount of a combination of sacubitril and valsartan in a 1:1 molar ratio for use in the prevention or treatment of a metabolic disease in a human patient.

In one embodiment thereof, the metabolic disease is manifested by at least one of the following criteria selected from adiposity, insulin resistance, impaired glucose metabolism, impaired glucose tolerance (IGT), conditions of impaired fasting plasma glucose, metabolic syndrome, diabetes mellitus type II; hyperglycemia, hyperinsulinaemia, hyperlipidaemia, dyslipidemia, and hypertriglyceridemia; in particular selected from insulin resistance, metabolic syndrome, and adiposity, in particular abdominal adiposity.

In a further embodiment thereof, the human patient also suffers from a cardiovascular disease. In one embodiment, the cardiovascular disease is selected from hypertension, chronic heart failure, acute heart failure, left ventricular dysfunction, endothelial dysfunction, diastolic dysfunction, ischemic cardiomyopathy, hypertrophic cardiomyopathy, diabetic cardiac myopathy, cardiac dysrhythmias, supraventricular and ventricular arrhythmias, atrial fibrillation, cardiac fibrosis, mitral stenosis and regurgitation, atrial flutter, detrimental vascular remodeling, heart target organ damage, plaque stabilization, myocardial infarction and its sequelae, atherosclerosis, angina pectoris, thrombosis, vascular aneurysm, vascular stenosis and infarction, vascular dementia, secondary aldosteronism, primary and secondary pulmonary hypertension, pulmonary congestion, pulmonary edema, right ventricular hypertrophy, and peripheral vascular disease, in particular from hypertension or heart failure, more particularly from chronic systolic heart failure with reduced ejection fraction (HF-rEF) or heart failure with preserved ejection fraction (HF-pEF).

In a further embodiment thereof, the pharmaceutical composition comprising the therapeutically effective amount of the combination of sacubitril and valsartan in a 1:1 molar ratio for use as defined herein is effective to induce at least one physiological effect selected from improving insulin sensitivity, ameliorating glucose metabolism, and increasing abdominal lipid mobilization in said patient suffers from a metabolic and cardiovascular disease. In one embodiment thereof, the patient suffers from hypertension and abdominal adiposity. In another embodiment thereof, the patient suffers from heart failure and abdominal adiposity.

Accordingly, in one aspect, the present invention relates to a pharmaceutical composition comprising a therapeutically effective amount or a prophylactically effective amount of a combination of sacubitril and valsartan in a 1:1 molar ratio for use in the prevention or treatment of a metabolic disease in a human patient, wherein the therapeutically effective amount of a combination of sacubitril and valsartan in a 1:1 molar ratio is effective to ameliorate glucose metabolism and to increase abdominal lipid mobilization in a patient suffering from a cardiovascular disease and adiposity. In one embodiment thereof, the patient suffers from hypertension and abdominal adiposity. In another embodiment thereof, the patient suffers from heart failure and abdominal adiposity.

In all of the aforementioned embodiments, the pharmaceutical composition comprises the therapeutically effective amount or a prophylactically effective amount of a combination of sacubitril and valsartan in a 1:1 molar ratio in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

In all of the aforementioned embodiments, the pharmaceutical composition comprises the therapeutically effective amount or a prophylactically effective amount of a combination of sacubitril and valsartan in a 1:1 molar ratio is administered to deliver the combination of sacubitril and valsartan in a 1:1 molar ratio twice daily with a dose of 50 mg, 100 mg, or 200 mg, wherein
a) the 50 mg dose of sacubitril and valsartan in a 1:1 molar ratio corresponds to 24 mg sacubitril and 26 mg valsartan,
b) the 100 mg dose of sacubitril and valsartan in a 1:1 molar ratio corresponds to 49 mg sacubitril and 51 mg valsartan, and
c) the 200 mg dose of sacubitril and valsartan in a 1:1 molar ratio corresponds to 97 mg sacubitril and 103 mg valsartan.

In one embodiment thereof, sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696), wherein
a) the 50 mg dose of sacubitril and valsartan in a 1:1 molar ratio corresponds to around 56.6 mg LCZ696,
b) the 100 mg dose of sacubitril and valsartan in a 1:1 molar ratio corresponds to around 113.1 mg LCZ696, and
c) the 200 mg dose of sacubitril and valsartan in a 1:1 molar ratio corresponds to around 226.2 mg LCZ696.

### Use of a Pharmaceutical Composition for the Manufacture of a Medicament

In another aspect, the present invention is directed to the use of a pharmaceutical composition comprising a therapeutically effective amount or a prophylactically effective amount of sacubitril and valsartan in a 1:1 molar ratio for the manufacture of a medicament fuse in the prevention or treatment of a metabolic disease in a human patient.

In one embodiment thereof, the metabolic disease is manifested by at least one of the following criteria selected from adiposity, insulin resistance, impaired glucose metabolism, impaired glucose tolerance (IGT), conditions of impaired fasting plasma glucose, metabolic syndrome, diabetes mellitus type II; hyperglycemia, hyperinsulinaemia, hyperlipidaemia, dyslipidemia, and hypertriglyceridemia; in particular selected from insulin resistance, metabolic syndrome, and adiposity, in particular abdominal adiposity.

In a further embodiment thereof, the human patient also suffers from a cardiovascular disease. In one embodiment, the cardiovascular disease is selected from hypertension, chronic heart failure, acute heart failure, left ventricular dysfunction, endothelial dysfunction, diastolic dysfunction, ischemic cardiomyopathy, hypertrophic cardiomyopathy, diabetic cardiac myopathy, cardiac dysrhythmias, supraventricular and ventricular arrhythmias, atrial fibrillation, cardiac fibrosis, mitral stenosis and regurgitation, atrial flutter, detrimental vascular remodeling, heart target organ damage, plaque stabilization, myocardial infarction and its sequelae, atherosclerosis, angina pectoris, thrombosis, vascular aneurysm, vascular stenosis and infarction, vascular dementia, secondary aldosteronism, primary and secondary pulmonary hypertension, pulmonary congestion, pulmonary edema, right ventricular hypertrophy, and peripheral vascular disease, in particular from hypertension or heart failure, more particularly from chronic systolic heart failure with reduced ejection fraction (HF-rEF) or heart failure with preserved ejection fraction (HF-pEF).

In a further embodiment thereof, the pharmaceutical composition comprising the therapeutically effective amount of the combination of sacubitril and valsartan in a 1:1 molar ratio is effective to induce at least one physiological effect selected from improving insulin sensitivity, ameliorating glucose metabolism, and increasing abdominal lipid mobilization in said patient suffers from a metabolic and cardiovascular disease. In one embodiment thereof, the patient suffers from hypertension and abdominal adiposity. In another embodiment thereof, the patient suffers from heart failure and abdominal adiposity.

Accordingly, in one aspect, the present invention relates to the use of a pharmaceutical composition comprising a therapeutically effective amount or a prophylactically effective amount of a combination of sacubitril and valsartan in a 1:1 molar ratio for the manufacture of a medicament for the prevention or treatment of a metabolic disease in a human patient, wherein the therapeutically effective amount of a combination of sacubitril and valsartan in a 1:1 molar ratio is effective to ameliorate glucose metabolism and to increase abdominal lipid mobilization in a patient suffering from a cardiovascular disease and adiposity. In one embodiment thereof, the patient suffers from hypertension and abdominal adiposity. In another embodiment thereof, the patient suffers from heart failure and abdominal adiposity.

In all of the aforementioned embodiments, the pharmaceutical composition comprises the therapeutically effective amount or a prophylactically effective amount of a combination of sacubitril and valsartan in a 1:1 molar ratio in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

In all of the aforementioned embodiments, the pharmaceutical composition comprises the therapeutically effective amount or a prophylactically effective amount of a combination of sacubitril and valsartan in a 1:1 molar ratio is administered to deliver the combination of sacubitril and valsartan in a 1:1 molar ratio twice daily with a dose of 50 mg, 100 mg, or 200 mg, wherein
a) the 50 mg dose of sacubitril and valsartan in a 1:1 molar ratio corresponds to 24 mg sacubitril and 26 mg valsartan,
b) the 100 mg dose of sacubitril and valsartan in a 1:1 molar ratio corresponds to 49 mg sacubitril and 51 mg valsartan, and
c) the 200 mg dose of sacubitril and valsartan in a 1:1 molar ratio corresponds to 97 mg sacubitril and 103 mg valsartan.

In one embodiment thereof, sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696), wherein
a) the 50 mg dose of sacubitril and valsartan in a 1:1 molar ratio corresponds to around 56.6 mg LCZ696,
b) the 100 mg dose of sacubitril and valsartan in a 1:1 molar ratio corresponds to around 113.1 mg LCZ696, and
c) the 200 mg dose of sacubitril and valsartan in a 1:1 molar ratio corresponds to around 226.2 mg LCZ696.

The following example is illustrative, but does not serve to limit the scope of the invention described herein.

### Example 1

A randomized, double-blind, parallel group study to evaluate metabolic effects of LCZ696 and amlodipine in obese hypertensive subjects (ClinicalTrials.gov Identifier: NCT01631864).

### Study Drug LCZ696:

LCZ696 refers to the supramolecular complex trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate]hemipentahydrate. This compound and pharmaceutical compositions thereof have been previously disclosed in WO2007/056546 and WO 2009/061713, whose preparative teachings are incorporated herein by reference.

LCZ696 is a first-in-class angiotensin receptor neprilysin inhibitor that comprises the molecular moieties of the NEP (neutral endopeptidase EC 3.4.24.11) inhibitor pro-drug AHU377 (N-(3-carboxy-1-oxopropyl)-(4S)-p-phenylphenylmethyl)-4-amino-(2R)-methylbutanoic acid ethyl ester) and the angiotensin receptor blocker valsartan as a single compound. AHU377 is metabolized by enzymatic cleavage to LBQ657 (N-(3-carboxy-1-oxopropyl)-(4S)-p-phenylphenylmethyl)-4-amino-(2R)-methylbutanoic acid), the active inhibitor of neutral endopeptidase, which is the major enzyme responsible for the breakdown of atrial natriuretic peptides.

### Comparator Drug Amlodipine:

Amlodipine or Amlodipine besylate can be purchased from commercial sources. Amlodipine is approved for the treatment of hypertension and coronary artery disease.

### Overall Study Design:

Ninety-eight (98) patients with mild-to-moderate hypertension and abdominal adiposity were randomized to LCZ696 400 mg or amlodipine (AML) 10 mg once daily for 8 weeks in a double-blind, double-dummy fashion. At baseline and week 8, peripheral insulin sensitivity (hyperinsulinemic-euglycemic glucose clamp), abdominal subcutaneous adipose tissue lipolysis (microdialysis), whole-body lipolysis (glycerol tracer kinetics), energy expenditure and substrate oxidation (indirect calorimetry) were determined.

### Study Patients

The study population included obese men and women (waist circumference ≥102 cm and ≥88 cm respectively), aged ≥18 years old, with elevated blood pressure either untreated (mean seated systolic blood pressure [msSBP] ≥30 mmHg and <180 mmHg at screening) or treated with up to two classes of antihypertensive therapy (msSBP ≤160 mmHg at screening and <180 mmHg at the end of the 4-week washout period). Females had to be of non-child bearing potential. Key exclusion criteria were severe hypertension (msDBP ≥100 mmHg and/or msSBP ≥180 mmHg at screening or at the end of the washout period), type 1 or type 2 diabetes mellitus (fasting plasma glucose ≥126 mg/dL or HbA_{1c}≥6.5%), dyslipidemia requiring pharmacological therapy, concomitant use of anti-hypertensives, anti-diabetics, or drugs that effects glucose or lipid metabolism, previous or current diagnosis of cardiac structural and functional abnormalities, history or current diagnosis of HF (NYHA Class II-IV), history of myocardial infarction, coronary bypass surgery or percutaneous coronary intervention during 6 months prior to screening, history of angioedema, or hypersensitivity to study drugs.

### Study Procedures:

### Study Design

This was a multicenter, randomized, double-blind, double-dummy, active-controlled, and parallel-group study. The study included an initial screening period of up to four weeks followed by a 4-week wash-out period and an 8-week randomized, double-blind treatment phase. Patients receiving anti-hypertensive medications at the time of screening discontinued the therapy during the washout period.

For the double-blind treatment period, patients were randomized to receive LCZ696 400 mg once daily (QD) or amlodipine 10 mg QD along with matching placebos for 8 weeks. Patients were also stratified into 4 groups based on the baseline Homeostasis Model Assessment of Insulin Resistance (HOMA-IR) (<2.5 and ≥2.5) and use of statin therapy within 8 weeks prior to randomization.

All participants provided informed consent prior to any study assessments. The study protocol was reviewed by the Independent Ethics Committee or Institutional Review Board for each center and the study was conducted in accordance with the ethical principles of the Declaration of Helsinki.

### Measurement of Insulin Sensitivity

The change in insulin sensitivity was measured from baseline to Week-8 for LCZ696 400 mg QD vs amlodipine 10 mg QD by a hyperinsulinemic-euglycemic glucose clamp (HEGC). The HEGC assessment was performed on Day −1 (baseline) and on Day 56 under fasting conditions. A dorsal hand vein was catheterized and kept warm for arterialized blood sampling and the contralateral arm was cathetered for the infusion of glucose and insulin. The procedure consisted of a 2-h primed infusion of insulin (a priming dose over the first 10 minutes [103 mU/m²/min at 0-5 minutes and 57 mU/m²/min at 5-10 minutes] followed by a continuous infusion at 40 mU/m²/min thereafter until 2-h) and a variable glucose infusion to achieve the steady state plasma insulin levels while maintaining the blood glucose levels at 90.1 mg/dL (5.0 mmol/L). Blood samples to determine glucose levels during the HEGC were collected continuously (automated clamp) or at approximately 5-min intervals (manual clamp). The last 30 minutes (minutes 90-120) of the clamp were considered as the steady state period and the mean glucose infusion rate was calculated for this time period.

The insulin sensitivity index (SI) was calculated from steady-state glucose infusion rates, and plasma insulin and glucose concentrations at steady-state (SI: Glucose infusion rate/[plasma glucose×plasma insulin], μg/kg*min/[mmol/L*pmol/L]). Furthermore whole-body glucose disposal rate (M, mg/min) was calculated from the mean glucose infusion rate at steady state to assess peripheral insulin sensitivity. Finally, the metabolic clearance rate (MCR) was calculated from M-value and the mean blood glucose concentration (BG) at steady-state (MCR: M/BG, min*mmol). The glucose disposal rate was also expressed per unit of insulin at steady-state, calculated from M-value and plasma insulin concentrations (M/l, U/min).

### Abdominal Subcutaneous Adipose Tissue Microdialysis

Assessment of lipolysis at rest was performed on Day 1 prior to first dosage and on Day 57. Abdominal subcutaneous adipose tissue lipolysis was assessed by microdialysis in the fasting condition. The microdialysis probe was placed in the subcutaneous adipose tissue 5-8 cm lateral from umbilicus under local anesthesia and sterile conditions, as described previously.²⁶ The process comprised a recovery phase of 60 minutes during which the probes were infused with perfusion solution (50 mM ethanol+T1 perfusion solution, p Dialysis AB, Sweden) at a flow rate of 0.3 μl/min followed by a flow calibration phase of 60 minutes. During the flow calibration phase, the perfusion rate was maintained at 0.3 μl/min for the first 30 minutes and thereafter was increased to 2.0 μl/min for the remaining 30 minutes. Microdialysate samples for measurement of the analytes were collected at 30 minutes and 45 minutes in the flow calibration phase.

Glycerol (an indicator of local lipolysis), glucose, and lactate concentrations were measured in the microdialysate, reflecting the interstitial concentrations in the adipose tissue. The ethanol ratio (ratio of ethanol concentration in dialysate to ethanol concentration in perfusate) was measured as an indicator of adipose tissue blood flow.

### Whole-Body Lipolysis

Whole-body lipolysis was estimated through measurement of [1,1,2,3,3-²H]-glycerol tracer kinetics. The assessments were made concurrently with the microdialysis procedure on Days 1 and 57. A glycerol tracer bolus was injected at the start of the microdialysis flow calibration phase, followed by a continuous infusion until the end of the sampling period. Blood samples were collected at 0, 30, 40, 50, and 60 minutes during the flow calibration phase and at 15, 30, and 45 minutes thereafter. The rate of appearance (Ra) of endogenous glycerol was calculated as the ratio of the glycerol tracer infusion rate to the plasma glycerol tracer enrichment. At steady state, glycerol Ra was calculated from glycerol tracer enrichment using Steele's equation.²⁷

### Energy Expenditure and Substrate Oxidation

Energy expenditure and substrate oxidation were assessed in parallel by indirect calorimetry using a ventilated hood system. The ventilated hood measurements were recorded for 30 minutes in the resting state (during the flow calibration phase). Resting energy expenditure was calculated using the abbreviated Weir equation²⁸ (REE=[3.9 (VO₂)+1.1 (VCO₂)] 1.44 kcal) (with VO2 and VCO2 expressed as L/min), provides an estimate of the amount of energy required by the body within 24 h at rest (i.e. an increase in REE with drug treatment suggest an increase in substrate oxidation at rest unrelated to energy demand).

### Blood Pressure Monitoring

Seating BP was monitored at screening and throughout the study using the same equipment. During the home stay period, patients were given a home measurement device and instructed to monitor BP twice weekly at approximately the same time each morning (7-9 AM).

### Safety Assessments

Safety assessments included adverse events (AEs) and serious adverse events (SAEs) and were conducted throughout the study by regular monitoring of hematology, blood chemistry and urine analysis as well as assessments of vital signs (BP and pulse measurements), ECG, physical condition and body weight.

### Statistical Methods

The pharmacodynamics (PD) analysis set included all patients with available PD data and no protocol deviations with relevant impact on PD data. The safety analysis set included all patients who received any of the study drugs.

It was calculated that a sample size of 90 completed subjects (45 in each group) would provide 80% power to detect a difference of 0.1 in SI at week-8 between the two treatments at 2.5% level of significance for a two-sided alternative. SI at 8 weeks was analyzed using analysis of covariance (ANCOVA) model with treatment as a fixed effect and baseline insulin sensitivity as a covariate for assessing the difference in mean effect of LCZ696 vs. amlodipine. For the purpose of a two-sided hypothesis of equality of treatment effects at 2.5% level of significance after the completion of the study, the point estimate and 97.5% CI for the difference along with the p-value for equality of two treatments were reported. The smaller level of significance (2.5%) was chosen to account for the conduct of an interim analysis. Differences between treatments for change in SI from baseline were analyzed as above.

For abdominal subcutaneous adipose tissue microdialysate data (ethanol ratio, dialysate lactate, dialysate glucose, and plasma glycerol, glycerol, NEFA, glucose, insulin, adrenalin, and noradrenalin) at rest, data was analyzed using repeated measures analysis on log transformed values with treatment, visit, time and treatment*visit*time interaction as fixed effects. Ratio to Day 1 (Day 57 vs. Day 1) at each treatment and ratio of LCZ696 vs amlodipine for ratio to Day 1 along with the corresponding 95% CIs and p-values are presented for 30 and 45 minute timepoints combined.

Oxidative metabolism was analyzed using an ANCOVA with treatment as fixed effect and baseline as covariate and the treatment mean difference with 95% CI and p-value are reported.

For biomarkers, data was analyzed using repeated measures analysis on log transformed values with treatment, visit, time and treatment*visit*time interaction as fixed effects. Ratio to Day 1 (Day 57 vs. Day 1) at each treatment and ratio of LCZ696 vs amlodipine for ratio to Day 1 along with the corresponding 95% CIs and p-values are presented.

### Results:

### Subjects

Of the 98 patients enrolled in the study, 92 patients (93.9%) completed the study. Discontinuations due to AEs occurred in two patients from the LCZ696 treatment group (one case of subarachnoid rupture of aneurysm and once case of pruritus) and three patients from the amlodipine group (one case each of nephrolithiasis, hypertension, and peripheral edema).

The patient demographics and baseline characteristics are presented in the following Table 1.

TABLE 1
Patient Demographics and Baseline Characteristics

| | LCZ696 400 mg | | Amlodipine | |
|---|---|---|---|---|
| Parameter | N = 50 | | N = 48 | |
| Age, years | 51.9 | (9.6) | 50.5 | (9.4) |
| Weight, kg | 101.2 | (17.3) | 104.3 | (14.1) |
| BMI, kg/m² | 32.6 | (4.6) | 33.3 | (4.4) |
| Gender, male, n (%) | 41 | (82) | 35 | (72.9) |
| Race, Caucasian, n (%) | 50 | (100) | 48 | (100) |
| Mean sitting SBP, mmHg | 141.7 | (11.15) | 139.7 | (12.62) |
| Mean sitting DBP, mmHg | 90.2 | (6.06) | 91.9 | (5.97) |
| Mean sitting pulse rate, bpm | 70.6 | (9.73) | 70.3 | (9.49) |

Data are mean (standard deviation) unless specified otherwise;
BMI, body mass index;
SBP, systolic blood pressure;
DBP, diastolic blood pressure;
bpm: beats per minute;

### Insulin Sensitivity

Plasma glucose and insulin profiles during the clamp were reflective of the procedure. Insulin reached steady state over the last 30 minutes of the clamp procedure.

Insulin sensitivity was assessed in 96 individuals.

LCZ696, but not amlodipine, was associated with a significant increase in SI (μg/kg*min/[mmol/L*pmol/L]) from baseline (mean [SD]: LCZ696, 1.63 [1.06]; AML, 1.69 [0.97]) to Day 56 (+0.192; 95% CI: 0.025, 0.359 and +0.065; 95% CI: −0.116; 0.246, respectively). However, the estimated treatment difference did not reach statistical significance (see Table 2).

TABLE 2
Mean Changes from Baseline in Insulin Sensitivity Variables Following
8 Weeks Treatment with LCZ696 and Amlodipine

| | | | |
|---|---|---|---|
| | LCZ696 | Amlodipine | Treatment Difference |
| Variable | 400 mg QD | 100 mg QD | (LCZ696-Amlodipine) |

Insulin sensitivity index (μg/kg * min/(mmol/L * pmol/L))

| | | | | | |
|---|---|---|---|---|---|
| Baseline (SD) | 1.63 | (1.06) | 1.69 | (0.97) | — |
| Day 56, adjusted mean (97.5% CI) | 1.86 | (1.69; 2.07) | 1.75 | (1.4; 1.96) | — |
| Adjusted mean change from baseline | 0.192 | (0.025; 0.359) | 0.065 | (−0.116; 0.246) | 0.128 (−0.155; 0.410) |
| (95% CI) | | | | | |

Glucose infusion rate by body weight (Mbw; mg/(min * kg))

| | | | | | |
|---|---|---|---|---|---|
| Baseline (SD) | 3.68 | (2.07) | 3.93 | (2.05) | — |
| Day 56, adjusted mean (97.5% CI) | 4.49 | (4.15; 4.82) | 3.87 | (3.51; 4.23) | — |
| Adjusted mean change from baseline | 0.64 | (0.30; 0.98) | 0.03 | (−0.33; 0.39) | 0.61 (0.12; 1.11) |
| (95% CI) | | | | | p = 0.016 |

Metabolic clearance rate (min * mmol).

| | | | | | |
|---|---|---|---|---|---|
| Baseline (SD) | 64.47 | (33.33) | 71.19 | (31.28) | — |
| Day 56, adjusted mean (97.5% CI) | 79.07 | (73.10; 85.05) | 69.42 | (63.03; 75.80) | — |
| Adjusted mean change from baseline | 10.74 | (4.77; 16.71) | 1.087 | (−5.29; 7.47) | 9.66 (0.89; 18.42) |
| (95% CI) | | | | | p = 0.0312 |

Importantly, LCZ696, but not amlodipine significantly increased the glucose infusion rate normalized by body weight (mg/min*kg) from baseline (3.68 [2.07] and 3.93 [2.05]) to Day 56 (mean change [95% CI]:+0.64 [0.30; 0.98] vs+0.03 [−0.33; 0.39]) which resulted in a significant treatment difference (p=0.016).

Furthermore, the glucose MCR (min*mmol) was significantly increased from baseline (64.47 [33.33] and 71.19 [31.28]) to Day 56 in the LCZ696 group versus amlodipine (+10.74 [4.77, 16.71] vs+1.087 (−5.29; 7.47); p=0.031).

### Abdominal Subcutaneous Adipose Tissue Microanalysis

An increase in glycerol from baseline to Day 57 was observed at all time-points in the LCZ696 group (adjusted geometric mean ratio [95% CI] at 30+45 min:1.05 [0.93; 1.18]), but the change did not reach the statistical significance. Compared with the amlodipine group, change from baseline in glycerol levels was significantly higher in the LCZ696 group at 45 min time-point on Day 57 (45 min:1.26 [1.04, 1.53], p=0.020; 30+45 min:1.22 [1.03, 1.45], p=0.026). (see Table 3)

TABLE 3
Adjusted Geometric Mean Ratios of Local Adipose Tissue Lipolysis
Variables Following 8 Weeks Treatment with LCZ696 and Amlodipine

| | | | |
|---|---|---|---|
| | | | Treatment |
| | | | differences, |
| | LCZ696 | Amlodipine | ratio of GMR |
| Time (min) | 400 mg QD | 10 mg QD | (95% CI) |

Glycerol (free) (μmol/L)

| | | | |
|---|---|---|---|
| 30 | 1.02 (0.90; 1.16) | 0.87 (0.76; 0.99) | 1.18 (0.96; 1.43) |
| | | p = 0.040 | |
| 45 | 1.07 (0.94; 1.22) | 0.85 (0.74; 0.98) | 1.26 (1.04; 1.53) |
| | | p = 0.024 | p = 0.020 |
| 30 + 45 | 1.05 (0.93; 1.18) | 0.86 (0.75; 0.97) | 1.22 (1.03; 1.45) |
| | | p = 0.018 | p = 0.026 |

Glucose (mmol/L)

| | | | |
|---|---|---|---|
| 30 | 1.03 (0.86; 1.20) | 1.00 (0.85; 1.16) | 1.03 (0.82; 1.29) |
| 45 | 1.01 (0.86; 1.18) | 0.98 (0.83; 1.16) | 1.03 (0.82; 1.29) |
| 30 + 45 | 1.02 (0.88; 1.18) | 0.99 (0.85; 1.16) | 1.03 (0.83; 1.27) |

Lactate (mmol/L)

| | | | |
|---|---|---|---|
| 30 | 0.81 (0.63; 1.04) | 0.91 (0.69; 1.19) | 0.89 (0.62; 1.29) |
| 45 | 0.86 (0.68; 1.09) | 0.90 (0.70; 1.16) | 0.96 (0.68; 1.36) |
| 30 + 45 | 0.84 (0.66; 1.06) | 0.90 (0.70; 1.16) | 0.93 (0.65; 1.31) |

Glucose and lactate levels did not differ significantly from baseline in any of the treatment groups and there was no difference between the treatment groups at Day 57 (Table 3).

No significant differences were observed between treatment groups for the change in ethanol ratio at any of the time points, except for 45 minute time point in the LCZ696 group. However, this increase was small and was not considered to be relevant for the analysis of the microdialysis experiment

### Whole-Body Lipolysis

The geometric mean rate of baseline endogenous glycerol appearance (95% CI) on Day 1 was 172.8 (158.0; 189.0) and 200.6 (182.4; 220.6) μmol/kg·min⁻¹ in the LCZ696 and amlodipine groups, respectively. Whole-body glycerol release (rate of appearance) was not significantly affected by amlodipine treatment, but was significantly lower at Day 57 versus Day 1 in those receiving LCZ696 (geometric mean ratio [95% CI], 0.93 [0.87, 1.00]; p=0.045)

### Energy Expenditure and Substrate Oxidation

At Day 1, the mean RQ (mean [SD] for LCZ696 and AML groups: 0.773 [0.063] and 0.768 [0.051]) and resting energy expenditure (2134.81 [397.98] kcal and 2180.30 [548.78] kcal) were comparable between groups. RQ and REE were not significantly different from baseline on Day 57 in either treatment group or between the treatment groups (mean between-group difference [95% CI]: RQ, 0.012 [−0.028; 0.051], p=0.557; REE, −31.26 (−149.75; 87.24) kcal, p=0.601). (see Table 4)

TABLE 4
Mean Changes from Baselline in Oxidative Metabolism Variables
Following 8 Weeks Treatment with LCZ696 and Amlodipine

| | | |
|---|---|---|
| | | Treatment Difference |
| LCZ696 400 mg QD | Amlodipine 10 mg QD | (LCZ696-Amlodipine) |

Respiratory quotient (carbon dioxide to oxygen ratio)

| | | | |
|---|---|---|---|
| Baseline mean (SD) | 0.77 (0.063) | 0.76 (0.051) | — |
| Day 57 adjusted mean | 0.78 (0.760; 0.814) | 0.77 (0.746; 0.804) | 0.01 (−0.028; 0.051) |
| (95% CI) | | | |

Resting energy expenditure (kcal)*

| | | | |
|---|---|---|---|
| Baseline mean (SD) | 2134.8 (397.98) | 2180.3 (548.78) | — |
| Day 57 adjusted mean | 1960.1 (1879.42; 2040.90) | 1991.4 (1904.82; 2078.01) | −31.2 (−149.75; 87.24)- |
| (95% CI) | | | |

### Blood Pressure

At baseline, msSBP (141.7±11.2 and 139.7±12.6 mmHg) and msDBP (90.2±6.1 and 91.9±5.9 mmHg) were comparable between the LCZ696 and amlodipine groups, respectively. At Day 57, the mean reductions in msSBP (−21.0±16.1 mmHg vs. −12.4±14.7 mmHg, respectively) and msDBP (−12.4±9.1 mmHg vs. −10.0±7.8 mmHg, respectively) were significantly higher in the LCZ696 compared to the amlodipine group, respectively.

### Safety Assessments

Of the 98 patients in the safety analysis set, 67 patients (68.4%) experienced at least one

AE. The overall incidence of AEs was lower in the LCZ696 group compared with the amlodipine group (60.0% vs. 77.1%). A total of 5 patients discontinued due to AEs: Two patients discontinued due to an SAE which was not suspected to be related to study drug (ruptured cerebral aneurysm in one patient in the LCZ696 group and nephrolithiasis in one patient in the amlodipine group); Three patients discontinued due to AEs suspected to be related to study drug (pruritis in one patient in the LCZ696 group, and single cases of hypertension and peripheral edema in the amlodipine group).

The most commonly occurring AEs were nasopharyngitis, peripheral edema and headache, each of which had an overall incidence of >10% (Table 2). AEs suspected to be study drug related occurred more frequently in the amlodipine group than in those receiving LCZ696 (46% vs. 24%). Peripheral edema, an AE known to be associated with amlodipine, was reported in 16 patients taking amlodipine and one patient taking LCZ696. Pruritus was reported by five patients, all of whom were in the LCZ696 group. Two patients in the LCZ696 group reported mild orthostatic hypertension, both of which resolved by the end of the study. Both of these events were suspected to be related to study drug. No deaths were reported in the study.

### Summary

LCZ696 and AML groups had comparable baseline characteristics with regards to age, blood pressure and body mass index (LCZ696 32.6±4.6 kg/m², AML 33.3±4.4 kg/m²). At week 8, the insulin sensitivity index trended towards improvement with LCZ696 (LCZ696 1.88 μg/kg*min/(mmol/L*pmol/L); AML 1.76 μg/kg*min/(mmol/L*pmol/L), p=0.128), and glucose infusion rate/body weight was significantly larger with LCZ696 (LCZ696 4.49 mg/min*kg; AML 3.88; p=0.0156). Subcutaneous adipose tissue lipolysis at rest increased with LCZ696, but decreased with AML (interstitial glycerol levels at week 8; LCZ696 80.53 μmol/L; amlodipine 63.99 μmol/L; p=0.003), while there were no relevant changes in ethanol ratio and glucose and lactate levels. The apparent exercise-induced increase in subcutaneous adipose tissue lipolysis was larger with LCZ696 compared to AML at 15 and 30 min of exercise (microdialysate glycerol at week 8; 15 min exercise: 153.1 μmol/L vs. 128.8 μmol/L, p=0.014; 30 min exercise: 187.63 μmol/L vs. 149.19 μmol/L, p=0.010). Whole-body lipolysis at rest and during exercise was not different between groups. While respiratory quotient increased with exercise in both treatment groups, energy expenditure and respiratory quotient at rest and during exercise did not differ between treatments.

### Conclusion

In conclusion, 8 weeks of treatment with LCZ696 improved insulin sensitivity and led to lipid mobilization from subcutaneous abdominal adipose tissue with unchanged whole body lipolysis and lipid oxidation at rest and during physical exercise in patients with hypertension and abdominal adiposity, thereby supporting for the first time the relevance of sustained elevation of neprilysin substrates such as natriuretic peptides in the context of AT1 receptor blockade to human glucose and lipid metabolism.

### EMBODIMENTS OF THE INVENTION

In particular, the present invention relates to the following embodiments:

### Embodiment 1

A method for the prevention or treatment of a metabolic disease in a human patient in need of such prevention or treatment comprising administering to said patient a pharmaceutical composition comprising a therapeutically effective amount or a prophylactically effective amount of a combination of sacubitril and valsartan in a 1:1 molar ratio.

### Embodiment 2

The method according to Embodiment 1, wherein the metabolic disease is manifested by at least one of the following criteria selected from adiposity, insulin resistance, impaired glucose metabolism, impaired glucose tolerance (IGT), conditions of impaired fasting plasma glucose, metabolic syndrome, diabetes mellitus type II; hyperglycemia, hyperinsulinaemia, hyperlipidaemia, dyslipidemia, and hypertriglyceridemia.

### Embodiment 3

The method according to Embodiment 1 or 2, wherein the metabolic disease is manifested by at least one of the following criteria selected from insulin resistance, metabolic syndrome, and adiposity, in particular abdominal adiposity.

### Embodiment 4

The method according to any one of the preceding embodiments, wherein sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

### Embodiment 5

The method according to any one of the preceding embodiments, wherein the human patient also suffers from a cardiovascular disease.

### Embodiment 6

The method according to Embodiment 5, wherein the cardiovascular disease is selected from hypertension, chronic heart failure, acute heart failure, left ventricular dysfunction, endothelial dysfunction, diastolic dysfunction, ischemic cardiomyopathy, hypertrophic cardiomyopathy, diabetic cardiac myopathy, cardiac dysrhythmias, supraventricular and ventricular arrhythmias, atrial fibrillation, cardiac fibrosis, mitral stenosis and regurgitation, atrial flutter, detrimental vascular remodeling, heart target organ damage, plaque stabilization, myocardial infarction and its sequelae, atherosclerosis, angina pectoris, thrombosis, vascular aneurysm, vascular stenosis and infarction, vascular dementia, secondary aldosteronism, primary and secondary pulmonary hypertension, pulmonary congestion, pulmonary edema, right ventricular hypertrophy, and peripheral vascular disease.

### Embodiment 7

The method according to Embodiment 6, wherein the patient suffers from hypertension or heart failure.

### Embodiment 8

The method according to Embodiment 7 wherein the patient suffers from heart failure, in particular chronic systolic heart failure with reduced ejection fraction (HF-rEF) or heart failure with preserved ejection fraction (HF-pEF).

### Embodiment 9

The method according to Embodiment 7, wherein the patient suffers from hypertension.

### Embodiment 10

The method according to any one of the preceding embodiments, wherein the therapeutically effective amount of the combination of sacubitril and valsartan in a 1:1 molar ratio is effective to induce at least one physiological effect selected from improving insulin sensitivity and increasing abdominal lipid mobilization in said patient.

### Embodiment 11

The method according to Embodiment 10, wherein the therapeutically effective amount of the combination of sacubitril and valsartan in a 1:1 molar ratio is effective to ameliorate glucose metabolism in said patient.

### Embodiment 12

The method according to Embodiment 10 or 11, wherein said patient suffers from hypertension and abdominal adiposity.

### Embodiment 13

The method according to Embodiment 10 or 11, wherein said patient suffers from heart failure and abdominal adiposity.

### Embodiment 14

The method according to any one of the preceding embodiments, wherein the therapeutically effective amount of the combination of sacubitril and valsartan in a 1:1 molar ratio is effective to ameliorate glucose metabolism and to increase abdominal lipid mobilization in a patient suffering from a cardiovascular disease and adiposity.

### Embodiment 15

The method according to Embodiment 14, wherein the patient suffers from hypertension and abdominal adiposity.

### Embodiment 16

The method according to any one of the preceding embodiments, wherein the pharmaceutical composition comprises in addition one or more pharmaceutically acceptable carriers.

### Embodiment 17

The method according to any one of the preceding embodiments, wherein the pharmaceutical composition is administered to deliver a daily overall dose of the combination of sacubitril and valsartan in a 1:1 molar ratio from about 50 mg to about 400 mg.

### Embodiment 18

The method according to any one of the preceding embodiments, wherein pharmaceutical composition is administered to deliver the combination of sacubitril and valsartan in a 1:1 molar ratio twice daily with a dose of 50 mg, 100 mg, or 200 mg.

### Embodiment 19

The method according to Embodiment 18, wherein
a) the 50 mg dose of sacubitril and valsartan in a 1:1 molar ratio corresponds to 24 mg sacubitril and 26 mg valsartan,
b) the 100 mg dose of sacubitril and valsartan in a 1:1 molar ratio corresponds to 49 mg sacubitril and 51 mg valsartan, and
c) the 200 mg dose of sacubitril and valsartan in a 1:1 molar ratio corresponds to 97 mg sacubitril and 103 mg valsartan.

### Embodiment 20

The method according to Embodiment 19, wherein sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696), and wherein
a) the 50 mg dose of sacubitril and valsartan in a 1:1 molar ratio corresponds to around 56.6 mg LCZ696,
b) the 100 mg dose of sacubitril and valsartan in a 1:1 molar ratio corresponds to around 113.1 mg LCZ696, and
c) the 200 mg dose of sacubitril and valsartan in a 1:1 molar ratio corresponds to around 226.2 mg LCZ696.

### Embodiment 21

A pharmaceutical composition comprising a therapeutically effective amount or a prophylactically effective amount of a combination of sacubitril and valsartan in a 1:1 molar ratio for use in the prevention or treatment of a metabolic disease in a human patient.

### Embodiment 22

The pharmaceutical composition for use according to Embodiment 21, wherein the metabolic disease is manifested by at least one of the following criteria selected from adiposity, insulin resistance, impaired glucose metabolism, impaired glucose tolerance (IGT), conditions of impaired fasting plasma glucose, metabolic syndrome, diabetes mellitus type II; hyperglycemia, hyperinsulinaemia, hyperlipidaemia, dyslipidemia, and hypertriglyceridemia.

### Embodiment 23

The pharmaceutical composition for use according to Embodiment 21 or 22, wherein the metabolic disease is manifested by at least one of the following criteria selected from insulin resistance, metabolic syndrome, and adiposity, in particular abdominal adiposity.

### Embodiment 24

The pharmaceutical composition for use according to any one of the preceding Embodiments 21 to 23, wherein sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

### Embodiment 25

The pharmaceutical composition for use according to any one of the preceding Embodiments 21 to 24, wherein the human patient also suffers from a cardiovascular disease.

### Embodiment 26

The pharmaceutical composition for use according to Embodiment 25, wherein the cardiovascular disease is selected from hypertension, chronic heart failure, acute heart failure, left ventricular dysfunction, endothelial dysfunction, diastolic dysfunction, ischemic cardiomyopathy, hypertrophic cardiomyopathy, diabetic cardiac myopathy, cardiac dysrhythmias, supraventricular and ventricular arrhythmias, atrial fibrillation, cardiac fibrosis, mitral stenosis and regurgitation, atrial flutter, detrimental vascular remodeling, heart target organ damage, plaque stabilization, myocardial infarction and its sequelae, atherosclerosis, angina pectoris, thrombosis, vascular aneurysm, vascular stenosis and infarction, vascular dementia, secondary aldosteronism, primary and secondary pulmonary hypertension, pulmonary congestion, pulmonary edema, right ventricular hypertrophy, and peripheral vascular disease.

### Embodiment 27

The pharmaceutical composition for use according to Embodiment 26, wherein the patient suffers from hypertension or heart failure.

### Embodiment 28

The pharmaceutical composition for use according to Embodiment 27, wherein the patient suffers from heart failure, in particular chronic systolic heart failure with reduced ejection fraction (HF-rEF) or heart failure with preserved ejection fraction (HF-pEF).

### Embodiment 29

The pharmaceutical composition for use according to Embodiment 27, wherein the patient suffers from hypertension.

### Embodiment 30

The pharmaceutical composition for use according to any one of the preceding Embodiments 21 to 29, wherein the therapeutically effective amount of the combination of sacubitril and valsartan in a 1:1 molar ratio is effective to induce at least one physiological effect selected from improving insulin sensitivity and increasing abdominal lipid mobilization in said patient.

### Embodiment 31

The pharmaceutical composition for use according to Embodiment 30, wherein the therapeutically effective amount of the combination of sacubitril and valsartan in a 1:1 molar ratio is effective to ameliorate glucose metabolism in said patient.

### Embodiment 32

The pharmaceutical composition for use according to Embodiment 30 or 31, wherein said patient suffers from hypertension and abdominal adiposity.

### Embodiment 33

The pharmaceutical composition for use according to Embodiment 30 or 31, wherein said patient suffers from heart failure and abdominal adiposity.

### Embodiment 34

The pharmaceutical composition for use according to Embodiment 21, wherein the therapeutically effective amount of the combination of sacubitril and valsartan in a 1:1 molar ratio is effective to ameliorate glucose metabolism and to increase abdominal lipid mobilization in a patient suffering from a cardiovascular disease and adiposity.

### Embodiment 35

The pharmaceutical composition for use according to Embodiment 34, wherein the patient suffers from hypertension and abdominal adiposity.

### Embodiment 36

The pharmaceutical composition for use according to any one of the preceding Embodiments 21 to 35, wherein the pharmaceutical composition comprises in addition one or more pharmaceutically acceptable carriers.

### Embodiment 37

The pharmaceutical composition for use according to any one of the preceding Embodiments 21 to 36, wherein the pharmaceutical composition is administered to deliver a daily overall dose of the combination of sacubitril and valsartan in a 1:1 molar ratio from about 50 mg to about 400 mg.

### Embodiment 38

The pharmaceutical composition for use according to any one of the preceding Embodiments 21 to 37, wherein pharmaceutical composition is administered to deliver the combination of sacubitril and valsartan in a 1:1 molar ratio twice daily with a dose of 50 mg, 100 mg, or 200 mg.

### Embodiment 39

The pharmaceutical composition for use according to Embodiment 38, wherein
a) the 50 mg dose of sacubitril and valsartan in a 1:1 molar ratio corresponds to 24 mg sacubitril and 26 mg valsartan,
b) the 100 mg dose of sacubitril and valsartan in a 1:1 molar ratio corresponds to 49 mg sacubitril and 51 mg valsartan, and
c) the 200 mg dose of sacubitril and valsartan in a 1:1 molar ratio corresponds to 97 mg sacubitril and 103 mg valsartan.

### Embodiment 40

The pharmaceutical composition for use according to Embodiment 39, wherein sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696), and wherein
a) the 50 mg dose of sacubitril and valsartan in a 1:1 molar ratio corresponds to around 56.6 mg LCZ696,
b) the 100 mg dose of sacubitril and valsartan in a 1:1 molar ratio corresponds to around 113.1 mg LCZ696, and
c) the 200 mg dose of sacubitril and valsartan in a 1:1 molar ratio corresponds to around 226.2 mg LCZ696.

### Embodiment 41

Use of a pharmaceutical composition comprising a therapeutically effective amount or a prophylactically effective amount of sacubitril and valsartan in a 1:1 molar ratio for the manufacture of a medicament fuse in the prevention or treatment of a metabolic disease in a human patient.

### Embodiment 42

Use according to Embodiment 41, wherein the metabolic disease is manifested by at least one of the following criteria selected from adiposity, insulin resistance, impaired glucose metabolism, impaired glucose tolerance (IGT), conditions of impaired fasting plasma glucose, metabolic syndrome, diabetes mellitus type II; hyperglycemia, hyperinsulinaemia, hyperlipidaemia, dyslipidemia, and hypertriglyceridemia.

### Embodiment 43

Use according to Embodiment 41 or 42, wherein the metabolic disease is manifested by at least one of the following criteria selected from insulin resistance, metabolic syndrome, and adiposity, in particular abdominal adiposity.

### Embodiment 44

Use according to any one of the preceding Embodiments 41 to 43, wherein sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

### Embodiment 45

Use according to any one of the preceding Embodiments 41 to 44, wherein the human patient also suffers from a cardiovascular disease.

### Embodiment 46

Use according to Embodiment 45, wherein the cardiovascular disease is selected from hypertension, chronic heart failure, acute heart failure, left ventricular dysfunction, endothelial dysfunction, diastolic dysfunction, ischemic cardiomyopathy, hypertrophic cardiomyopathy, diabetic cardiac myopathy, cardiac dysrhythmias, supraventricular and ventricular arrhythmias, atrial fibrillation, cardiac fibrosis, mitral stenosis and regurgitation, atrial flutter, detrimental vascular remodeling, heart target organ damage, plaque stabilization, myocardial infarction and its sequelae, atherosclerosis, angina pectoris, thrombosis, vascular aneurysm, vascular stenosis and infarction, vascular dementia, secondary aldosteronism, primary and secondary pulmonary hypertension, pulmonary congestion, pulmonary edema, right ventricular hypertrophy, and peripheral vascular disease.

### Embodiment 47

Use according to Embodiment 46, wherein the patient suffers from hypertension or heart failure.

### Embodiment 48

Use according to Embodiment 47, wherein the patient suffers from heart failure, in particular chronic systolic heart failure with reduced ejection fraction (HF-rEF) or heart failure with preserved ejection fraction (HF-pEF).

### Embodiment 49

Use according to Embodiment 47, wherein the patient suffers from hypertension.

### Embodiment 50

Use according to any one of the preceding Embodiments 41 to 49, wherein the therapeutically effective amount of the combination of sacubitril and valsartan in a 1:1 molar ratio is effective to induce at least one physiological effect selected from improving insulin sensitivity and increasing abdominal lipid mobilization in said patient.

### Embodiment 51

Use according to Embodiment 50, wherein the therapeutically effective amount of the combination of sacubitril and valsartan in a 1:1 molar ratio is effective to ameliorate glucose metabolism in said patient.

### Embodiment 52

Use according to Embodiment 50 or 51, wherein said patient suffers from hypertension and abdominal adiposity.

### Embodiment 53

Use according to Embodiment 50 or 51, wherein said patient suffers from heart failure and abdominal adiposity.

### Embodiment 54

Use according to Embodiment 51, wherein the therapeutically effective amount of the combination of sacubitril and valsartan in a 1:1 molar ratio is effective to ameliorate glucose metabolism and to increase abdominal lipid mobilization in a patient suffering from a cardiovascular disease and adiposity.

### Embodiment 55

Use according to Embodiment 54, wherein the patient suffers from hypertension and abdominal adiposity.

### Embodiment 56

Use according to any one of the preceding Embodiments 41 to 55, wherein the pharmaceutical composition comprises in addition one or more pharmaceutically acceptable carriers.

### Embodiment 57

Use according to any one of the preceding Embodiments 41 to 56, wherein the pharmaceutical composition is administered to deliver a daily overall dose of the combination of sacubitril and valsartan in a 1:1 molar ratio from about 50 mg to about 400 mg.

### Embodiment 58

Use according to any one of the preceding Embodiments 41 to 57, wherein pharmaceutical composition is administered to deliver the combination of sacubitril and valsartan in a 1:1 molar ratio twice daily with a dose of 50 mg, 100 mg, or 200 mg.

### Embodiment 59

Use according to Embodiment 58, wherein
a) the 50 mg dose of sacubitril and valsartan in a 1:1 molar ratio corresponds to 24 mg sacubitril and 26 mg valsartan,
b) the 100 mg dose of sacubitril and valsartan in a 1:1 molar ratio corresponds to 49 mg sacubitril and 51 mg valsartan, and
c) the 200 mg dose of sacubitril and valsartan in a 1:1 molar ratio corresponds to 97 mg sacubitril and 103 mg valsartan.

### Embodiment 60

Use according to Embodiment 59, wherein sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate

(LCZ696), and wherein
a) the 50 mg dose of sacubitril and valsartan in a 1:1 molar ratio corresponds to around 56.6 mg LCZ696,
b) the 100 mg dose of sacubitril and valsartan in a 1:1 molar ratio corresponds to around 113.1 mg LCZ696, and
c) the 200 mg dose of sacubitril and valsartan in a 1:1 molar ratio corresponds to around 226.2 mg LCZ696.

### REFERENCES

Beleigoli 2009: Beleigoli A M, Diniz M F, Ribeiro A L (2009). Natriuretic peptides: linking heart and adipose tissue in obesity and related conditions—a systematic review. Obes Rev; 10:617-26.
Birkenfeld et al 2005: Birkenfeld A L, Boschmann M, Moro C, et al (2005). Lipid Mobilization with Physiological Atrial Natriuretic Peptide Concentrations in Humans. J Clin Endocrinol Metab 90: 3622-3628.
Birkenfeld et al 2008: Birkenfeld A L, Budziarek P, Boschmann M, Moro C, Adams F, Franke G, Berlan M, Marques M A, Sweep F C, Luft F C, Lafontan M and Jordan J. Atrial natriuretic peptide induces postprandial lipid oxidation in humans. Diabetes. 2008; 57:3199-204.
Birkenfeld et al. 2012: Birkenfeld A L, Boschmann M, Engeli S, Moro C, Arafat A M, Luft F C and Jordan J. Atrial natriuretic peptide and adiponectin interactions in man. PLoS One. 2012; 7:e43238.
Engeli et al. 2012: Engeli S, Birkenfeld A L, Badin P M, Bourlier V, Louche K, Viguerie N, Thalamas C, Montastier E, Larrouy D, Harant I, de Glisezinski I, Lieske S, Reinke J, Beckmann B, Langin D, Jordan J and Moro C. Natriuretic peptides enhance the oxidative capacity of human skeletal muscle. J Clin Invest. 2012; 122:4675-9
Heinisch 2011: Heinisch B B, Vila G, Resl M, et al (2011). B-type natriuretic peptide (BNP) affects the initial response to intravenous glucose: a randomised placebo-controlled cross-over study in healthy men. Diabetologia; 55:1400-5.
Magnusson et al 2012: Magnusson M, Jujic A, Hedblad B, Engstrom G, Persson M, Struck J, Morgenthaler N G, Nilsson P, Newton-Cheh C, Wang T J and Melander O. Low plasma level of atrial natriuretic peptide predicts development of diabetes: the prospective Malmo Diet and Cancer study. J Clin Endocrinol Metab. 2012; 97:638-45.
McMurray 2010: McMurray J J, Holman R R, Haffner S M, et al. NAVIGATOR Study Group Effect of valsartan on the incidence of diabetes and cardiovascular events. N Engl J Med 2010; 362:1477-1490.
Miyashita et al 2009: Miyashita K, Itoh H, Tsujimoto H, et al (2009). Natriuretic Peptides/cGMP/cGMP-Dependent Protein Kinase Cascades Promote Muscle Mitochondrial Biogenesis and Prevent Obesity. Diabetes. 58:2880-92
Rubattu 2008: Rubattu S, Sciarretta S, Valenti V, Stanzione R and Volpe M. Natriuretic peptides: an update on bioactivity, potential therapeutic use, and implication in cardiovascular diseases. Am J Hypertens. 2008; 21:733-41.
Sengenes et al 2000: Sengenes C, Berlan M, De Glisezinski I, et al (2000). Natriuretic peptides: a new lipolytic pathway in human adipocytes. FASEB J; 14:1345-51.
Souza et al 2011: Souza S C, Chau M D, Yang Q, et al (2011). Atrial natriuretic peptide regulates lipid mobilization and oxygen consumption in human adipocytes by activating AMPK. Biochem Biophys Res Commun; 410:398-403.
Wang 2007: Wang T J, Larson M G, Keyes M J et al (2007). Association of Plasma Natriuretic Peptide Levels With Metabolic Risk Factors in Ambulatory Individuals. Circulation; 115:1345-1353

1-15. (canceled)

16. A method for the prevention or treatment of a metabolic disease in a human patient in need of such prevention or treatment comprising administering to said patient a pharmaceutical composition comprising a therapeutically effective amount or a prophylactically effective amount of a combination of sacubitril and valsartan in a 1:1 molar ratio, wherein the therapeutically effective amount of the combination of sacubitril and valsartan in a 1:1 molar ratio is effective to induce at least one physiological effect selected from improving insulin sensitivity, increasing abdominal lipid mobilization and ameliorating glucose metabolism in said patient.

17. The method according to claim 16, wherein the therapeutically effective amount of the combination of sacubitril and valsartan in a 1:1 molar ratio is effective to induce at least one physiological effect selected from improving insulin sensitivity and increasing abdominal lipid mobilization in said patient.

18. The method according to claim 18, wherein the therapeutically effective amount of the combination of sacubitril and valsartan in a 1:1 molar ratio is effective to ameliorate glucose metabolism in said patient.

19. The method according to claim 16, wherein the metabolic disease is manifested by at least one of the following criteria selected from adiposity, insulin resistance, and metabolic syndrome.

20. The method according to claim 19, wherein the metabolic disease is manifested by abdominal adiposity.

21. The method according to claim 16, wherein sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ696).

22. The method according to claim 16, wherein the human patient also suffers from a cardiovascular disease.

23. The method according to claim 22, wherein the patient suffers from hypertension or heart failure.

24. The method according to claim 23, wherein the patient suffers from heart failure, in particular chronic systolic heart failure with reduced ejection fraction (HF-rEF) or heart failure with preserved ejection fraction (HF-pEF).

25. The method according to claim 23, wherein the patient suffers from hypertension.

26. The method according to claim 25, wherein said patient suffers from hypertension and abdominal adiposity.

27. The method according to claim 23, wherein said patient suffers from heart failure and abdominal adiposity.

28. The method according to claim 16 wherein the therapeutically effective amount of the combination of sacubitril and valsartan in a 1:1 molar ratio is effective to ameliorate glucose metabolism and to increase abdominal lipid mobilization in a patient suffering from a cardiovascular disease and adiposity.

29. The method according to claim 25, wherein the pharmaceutical composition comprises in addition one or more pharmaceutically acceptable carriers.

30. The method according to claim 18, wherein the pharmaceutical composition is administered to deliver a daily overall dose of the combination of sacubitril and valsartan in a 1:1 molar ratio from about 50 mg to about 400 mg.

31. The method according to claim 30, wherein pharmaceutical composition is administered to deliver the combination of sacubitril and valsartan in a 1:1 molar ratio twice daily with a dose of 50 mg, 100 mg, or 200 mg.

32. The method according to claim 31, wherein
a) the 50 mg dose of sacubitril and valsartan in a 1:1 molar ratio corresponds to 24 mg sacubitril and 26 mg valsartan,
b) the 100 mg dose of sacubitril and valsartan in a 1:1 molar ratio corresponds to 49 mg sacubitril and 51 mg valsartan, and
c) the 200 mg dose of sacubitril and valsartan in a 1:1 molar ratio corresponds to 97 mg sacubitril and 103 mg valsartan.

33. The method according to claim 31, wherein sacubitril and valsartan in a 1:1 molar ratio are delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate (LCZ896), and wherein
a) the 50 mg dose of sacubitril and valsartan in a 1:1 molar ratio corresponds to around 56.6 mg LCZ696,
b) the 100 mg dose of sacubitril and valsartan in a 1:1 molar ratio corresponds to around 113.1 mg LCZ696, and
c) the 200 mg dose of sacubitril and valsartan in a 1:1 molar ratio corresponds to around 226.2 mg LCZ696.
